(19) United States
(12) Patent Application Publication
(10) Pub. No.: US 2012/0265183 A1
(43) Pub. Date: Oct. 18, 2012

(54) CATHETER SYSTEM FOR BYPASS SURGERY AND A METHOD FOR PREPARING A CATHETER SYSTEM FOR BYPASS SURGERY

(75) Inventors: Cornelis Antonius Franciscus Tulleken, Utrecht (NL); Theodorus Petrus Cornelis Van Doormaal, De Bilt (NL)

(73) Assignee: AMJ bv, Utrecht (NL)

(21) Appl. No.: 13/395,358

(22) PCT Filed: Sep. 11, 2009
(86) PCT No.: PCT/NL2009/000178

§ 371 Date: Jun. 26, 2012

(51) Int. Cl. A61B 18/20

(52) U.S. Cl. 606/10

(57) ABSTRACT

A catheter system is provided comprising a laser catheter with a fibre bundle of optical fibres emitting a light beam in the distal direction of the catheter and a laser apparatus, comprising one or more lasers for supplying light to the optical fibres. The catheter system is preset or adjusted for emitting a pulsating light beam with an ablation power of at least 40 mJ/mm2 per pulse at the location of a light emitting surface. A method for preparing such a catheter system is provided comprising the step of a) measuring said ablation power at the location of the light emitting surface; b) comparing the measured ablation power with a predefmed power value requirement; c) in case the measured ablation power is different from the predefmed power value requirement, adjusting the ablation power of the pulsating light beam to meet the predefined power value requirement.

### FIELD OF THE INVENTION

The present invention relates to a catheter system and more particularly to a catheter system comprising a laser catheter provided with a fibre bundle of optical fibres having distal ends defining a light emitting surface for emitting a light beam in the distal direction of the catheter and a laser apparatus, comprising one or more lasers for supplying light to the optical fibres.

### BACKGROUND OF THE INVENTION

A laser catheter for bypass surgery is known from EP 750,476. This document describes the use in the ELANA (Excimer Laser Assisted Non-occlusive Anastomosis) operating technique. For this technique, one requires a catheter and a ring, which are jointly called Elana® Arteriotomy System.

The catheter disclosed in EP 750,476 is used for performing an ETS-anastomosis (ETS=End To Side) between a graft vessel and a target vessel. The graft is fixed with an end to the side of the target vessel, while the blood flow through the target vessel, also called recipient vessel, is not interrupted, i.e. blood continues to flow through the target vessel while performing the anastomosis. For this purpose, first the graft vessel is fixed to the target vessel and subsequently, after this fixation is established, the flow connection between the target vessel and graft vessel is made by removing the part of the wall of the target vessel which lies in front of the fixed end of the graft vessel. Said part of the wall of the target vessel is removed by means of a tubular arrangement of optical fibres emitting a tubular bundle of laser light beam originating from the fibres and a suction gripper provided inside the tubular arrangement of optical fibres. The tubular bundle of the laser light beam ablates a circle into the wall of the target vessel, resulting in a circular passage connecting the lumens of the graft vessel and target vessel. The circular wall part of the target vessel—i.e. the part lying inside said circle—is gripped by the suction gripper and removed together with the withdrawal of the catheter after the ablation operation. The distal ends of the optical fibres of this known laser catheter define a circle extending in a plane essentially perpendicular to the longitudinal axis of the catheter. During use the laser catheter extends perpendicular to the target vessel, resulting in a perpendicular ETS-anastomosis with a circular passage between the graft vessel and target vessel. When applying this technique, it is important that the circular wall part of the target vessel is removed completely during operation. If not, additional actions have to be taken to prevent the circular wall part from entering the target vessel or from obstructing the blood flow.

### SUMMARY OF THE INVENTION

The object of the present invention is to provide a laser catheter for bypass surgery, such as especially for the ELANA technique, that will increase the effectiveness of the operation, which is defined as the percentage of complete cut of the so called ‘flap’. The flap is the wall part (of the target vessel) which is to be cut away from the wall of the target vessel for providing the connection from the target vessel to the graft vessel.

According to a first aspect, this is achieved by providing a catheter system comprising a laser catheter provided with a fibre bundle of optical fibres having distal ends defining a light emitting surface for emitting a light beam in the distal direction of the catheter, and a laser apparatus, comprising one or more lasers for supplying light to the optical fibres, wherein the system is preset or adjusted for emitting a pulsating light beam with an ablation power of at least 40 mJ/mm² per pulse at the location of the light emitting surface.

In former techniques, the power is normally limited to 20 mJ/mm² or less to prevent the light beam from cutting through the whole target vessel, i.e. through both sides of the vessel. Emitting the light bundle of pulsating light beam from the light emitting surface with an ablation power of at least 40 mJ/mm² per pulse or more preferably at least 45 mJ/mm² per pulse, results in a more reliable and better cutting away of tissue, which means that the flap is cut away more effectively.

The area used for calculating the ablation power is the area of the light emitting surface, this being the sum of all the distal end areas of all optical fibres. The ablation power is measured at the location of the light emitting surface, i.e. the energy of the light beam actually emitted by the distal ends of the optical fibres is measured.

Surprisingly, despite the higher ablation power, cutting through both sides of the vessel does not occur, only the vessel wall directly adjacent the target vessel and light emitting surface is cut. It is assumed that the blood flowing through the vessel adequately prevents the light beam form cutting through the vessel at the opposite site. The higher ablation power of at least 40 mJ/mm² per pulse further guarantees that the flap is fully cut away without leaving any remnants connected to the target vessel.

According to a second aspect, the object of the invention is achieved by providing a catheter system comprising a laser catheter provided with a fibre bundle of optical fibres having distal ends defining a light emitting surface for emitting a pulsating light beam in the distal direction of the catheter, a laser apparatus, comprising one or more lasers for supplying light to the optical fibres, and an adjuster for setting an ablation power of the pulsating light beam, a power sensor for measuring the ablation power and for generating a power signal representative for the measured ablation power and a controller for controlling the adjuster in response to the power signal to set said ablation power at least 40 mJ/mm² per pulse at the location of the light emitting surface.

The ablation power at the location of the light emitting surface is among other factors dependent on the attenuation characteristics of the optical fibres used. The fibre bundles of optical fibres are typically used only once or a few times, while the laser apparatus is used many times and thus with several different fibre bundles. It may therefore be advantageous to adjust the ablation power for each combination of a laser apparatus and a fibre bundle of optical fibres.

The laser apparatus may be for example an excimer laser which provides ultraviolet light. This ultraviolet light may have a wavelength in the range of 240-340, or about 308 nm.

In a further embodiment of the invention, the ablation power is in the range of 40-100 mJ/mm² per pulse, preferably in the range of 40-60 mJ/mm² per pulse. An advantage of an ablation power in this range is that the so called ‘flap’ is cut away very effectively. Increasing the ablation power beyond this range on one hand may have disadvantageous effects, for example on the blood flowing through the target vessel, while on the other hand the effectiveness of cutting away the flap does not appear to improve further.

In another embodiment, the optical fibres of the fibre bundle are arranged in a tubular configuration to define said light emitting surface as being ring shaped. Since an ETS-anastomosis (ETS=End To Side) is usually done with a tubular shaped graft, it may be advantageous to provide a fibre bundle arranged in a tubular configuration for easy insertion. The ring shaped light emitting surface will result in the flap having a circular shape, and therefore the connecting opening between the graft wall and the target wall will also be circular.

In another embodiment, the catheter system further comprises a timing device for setting emitting intervals and break intervals at predefined time values, wherein the catheter system is arranged to emit the pulsating light beam during at least two emitting intervals, wherein each two consecutive intervals are separated by a break interval, during which emitting the pulsating light beam is interrupted.

The dissipation of energy at the surface of the target vessel may create gasses. These gasses may cause the surface of the target vessel wall to move slightly. An advantage of emitting the pulsating light beam during at least two emitting intervals, separated by a break interval, is that during the break interval the surface of the target vessel can stretch back to its preferred position. It is known that cutting is performed most effectively when the surface of the target vessel is positioned adjacent to and stretched along the light emitting surface. Allowing the surface of the target to stretch back will increase the effectiveness of the cutting in the consecutive emitting interval. Also gasses created by the ablation can disperse or disappear during the break interval and an abundance or built up of gasses is prevented.

In a further embodiment the timing device further comprises an input device for inputting predefined time values for the emitting intervals and break intervals. In preferred embodiments, the emitting intervals are at least 2 s, or preferable in the range of 2-5 s. The break intervals may be at least 1 s or preferably in the range of 1-5 s.

In another embodiment the pulsating light beam has a pulsating frequency of at least 25 Hz, or preferably in the range of 25-80 Hz, or more preferably around 40 Hz. An advantage of providing a pulsating light beam may be that with a pulsating light beam high energies in a short time can dissipate, resulting in cutting tissue, without heating up a larger part of the tissue. Such heating could cause burning damages.

According to another embodiment of the catheter system, the pulsating light beam has a pulse width in the range of 50-300 ns at Full Width at Half Maximum (F.W.H.M). Also short pulses may result advantageously in high energy dissipation without ablation damages to the vessels.

In order to remove the flap of the target vessel after ablation of the ring of tissue, it is advantageous to provide the distal part of the catheter with a gripper for gripping tissue inside the tubular bundle of the light beam. According to the invention, the gripper preferably comprises a hollow channel extending within the fibre bundle of optical fibres and connectable to a vacuum source, wherein the distal end of the channel defines a suction mouth. In order to ensure a firm gripping of the cut out part of the wall tissue, it is according to the invention advantageous to arrange the suction mouth at a distance proximally from the light emitting surface and so that it defines a suction surface parallel to the light emitting surface. The suction surface parallel to the light emitting surface ensures an easy gripping of the cut out part, i.e. the flap, all over its surface.

The stop surface forms locally a radial bulge on the outer surface of the catheter. When the catheter is inserted into a graft vessel, this bulge will be visible as a bulge in the wall of the graft vessel or at least tangible with the fingers of the surgeon. This enables the surgeon to control or correct the orientation and the position of the light emitting surface.

In another embodiment of the invention, the catheter system further comprises a ring member having dimensions adapted for, on the one hand, insertion of the distal end of the fibre bundle of optical fibres through said ring member and for, on the other hand, preventing passage of the stop surface through said ring member.

In a further embodiment, the catheter system comprises a graft vessel having diameter dimensions allowing, on the one hand, passage of the laser catheter and, on the other hand, insertion through said ring member. This graft vessel can be a donor vessel obtained from the patient or another person, but it can also be an artificial graft vessel manufactured from biological and/or non-biological material.

Before connecting the graft vessel to the target vessel, the graft vessel will be prepared for the bypass procedure by inserting one end of the graft vessel through the ring member and folding back the end of the graft vessel around the ring member. Before using the laser catheter, this folded end of the graft vessel with the ring member located in the inside of the fold, will be attached to the wall of the target vessel. Subsequently, when the laser catheter has been introduced into the graft vessel and the laser operation is performed, the ring member will prevent the laser catheter from advancing too far into the target vessel as soon as the stop surface comes to rest onto the ring member.

In a further embodiment in which the one end of the graft vessel is inserted through said ring member and folded back over said ring member, the angle between the graft vessel and the part of the graft vessel folded back is in the range of 90-180 degrees. It can be advantageous to fold a part of the graft vessel. In that way it can easily be connected to the target vessel.

According to a further aspect, the invention relates to a method for preparing a catheter system according to the invention. The advantages of the method follow from the preceding.

### BRIEF DESCRIPTION OF THE DRAWINGS

Further advantageous embodiments of the assembly according to the invention are described in the claims and in the following description with reference to the drawing, in which:

FIG. 1 shows a laser catheter of a catheter system according to the invention, wherein FIG. 1A is a longitudinal view in cross section and FIG. 1B shows an end view according to arrow IB in FIG. 1A.

FIG. 2 shows a part of a catheter system according to the invention and a sequence of steps in a ETS-anastomosis procedure according to the invention, wherein FIG. 2 is sub-divided into the FIGS. 2a, 2b, 2c and 2d, which each show a different step.

FIG. 3 shows a schematic overview of the devices involved with manipulating the laser light.

FIGS. 4a/b show a power output diagram of the pulsating light beam.

### DETAILED DESCRIPTION OF THE INVENTION

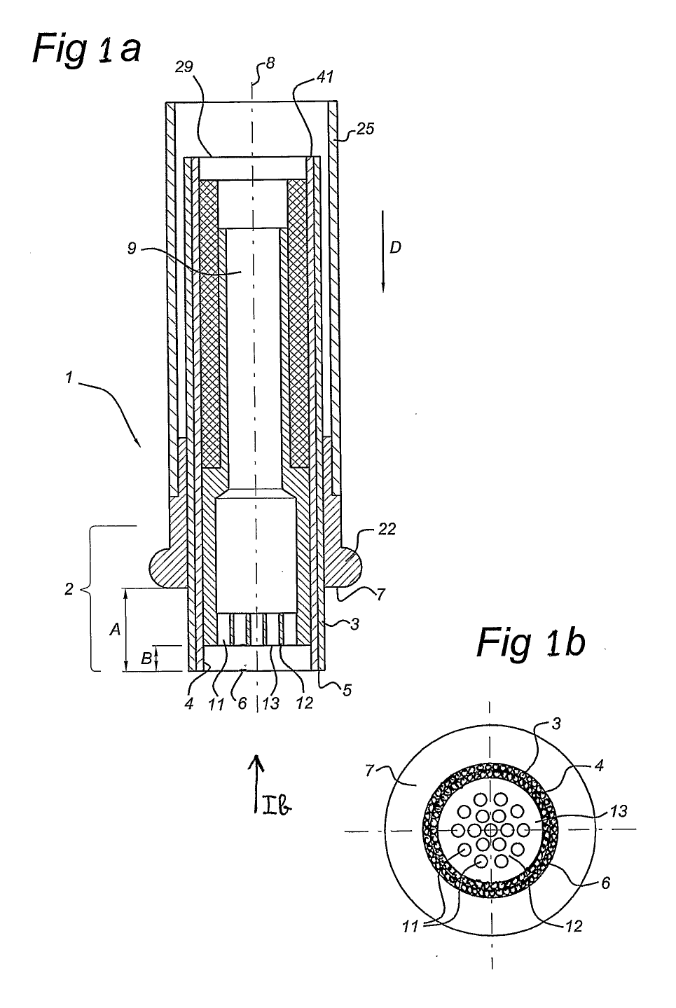

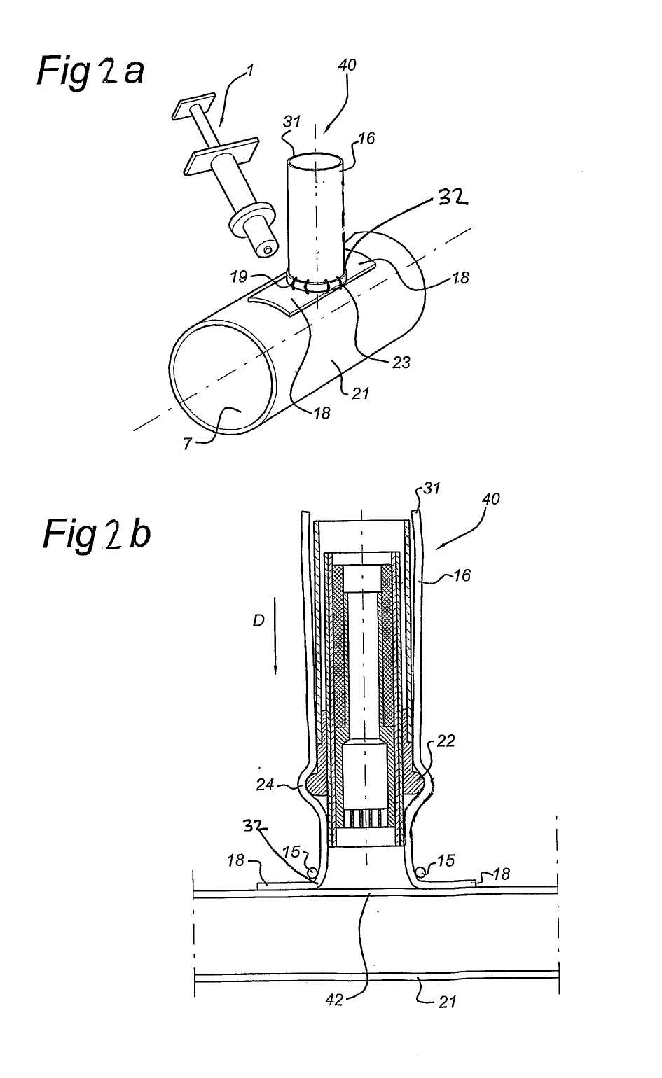

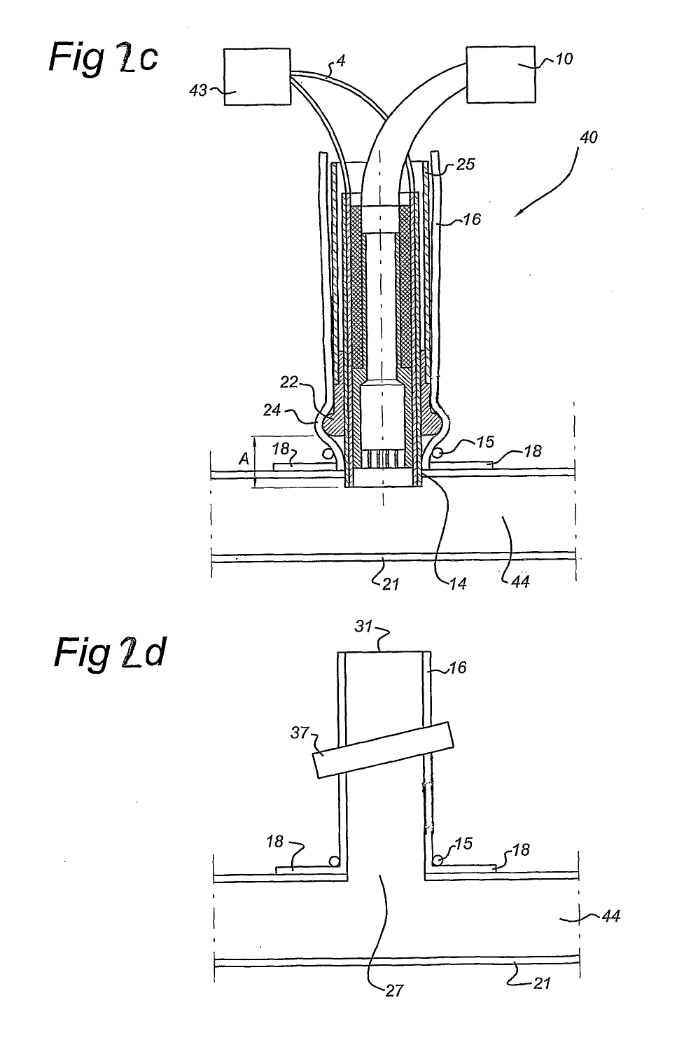

FIG. 1 shows a laser catheter 1. The distal part 2 of the laser catheter 1 is provided with a fibre bundle arrangement 3 of optical fibres 4. The optical fibres 4 have distal ends 5, which together define a light emitting surface 6. When a pulsating laser source is.connected to the proximal ends 41 of the optical fibres 4, a pulsating light beam will emit from each of these distal ends 5 of the optical fibres 4. The distal ends of the optical fibres 4 extend parallel to the longitudinal axis 8 of the catheter, so that the emitted light beam will extend parallel to the longitudinal axis 8 in the distal direction indicated by arrow D. When the fibre bundle comprises a tubular bundle of optical fibres, the light emitting surface will be ring-shaped and the pulsating light beam bundle will result in a tubular light beam in the distal direction D of the catheter. However, other arrangement of optical fibres are also possible, for example with optical fibres randomly distributed in ring-shape or a massive fibre bundle of fibres. The latter would enable the ablation of a complete disc—or another shape—without leaving a core. In the following an embodiment is described with a tubular bundle of optical fibres.

The laser catheter 1 further comprises a casing 25 surrounding the tubular fibre bundle 3 of optical fibres. The tubular fibre bundle 3 of optical fibres 4 encloses a channel 9. The proximal end 29 of the channel 9 can be connected to a vacuum source 10 (see FIG. 2c) in order to apply a suction force to the channel 9. The distal end of the channel 9 is provided with a plate, defining the suction surface 13 and provided with suction apertures 11. The distal end of the channel 9 thus forms a suction mouth 12, which acts as a gripper when vacuum is applied at the proximal end 29 of channel 9. The suction mouth 11 is provided at a distance B proximally from the light emitting surface 6. This distance B will at least be about the thickness of the wall of the target vessel (21, in FIG. 2).

However, in some cases, for example if a complete disc of tissue is to be cut away, there is no need for the use of a gripper.

The distal end of the casing 25 is provided with a radial bulge 22. The distal side of the radial bulge 22 forms a stop surface 7. This stop surface lies proximally at a distance A from the light emitting surface 6. This distance A will at least be about twice the wall thickness of the first vessel 16 (which follows from FIG. 2c discussed below) plus the wall thickness of the target vessel 21 (which also follows from FIG. 2c).

In order to ensure a good gripping of the flap 14 (FIG. 2c)—the term ‘flap’ indicates ‘the tissue part separated after ablation of a piece of tissue’—by the suction mouth 12, the suction surface 13 of the suction mouth extends parallel to the light emitting surface 6.

The bulge 22 with the stop surface 7 also extends parallel to the light emitting surface 6. It will be clear that the bulge 22 is preferably a bulge extending continuously around the catheter, but that it may also be a discontinuous bulge. The outer diameter of the bulge 22 is larger than the inner diameter of the ring member 15. This reliably prevents the distal part of the catheter from being inserted too far into the target vessel.

The distal ends 5 of the optical fibres 4 lie closely packed together with the longitudinal walls of adjacent fibres against each other to form together a tubular arrangement 3 having a circular cross-section as can be seen in FIG. 1B. However, with different arrangement of optical fibres, differently shaped cross-sections are also possible. The distal end faces of all the optical fibres 4 together define an essentially flat light emitting surface 6, which extends perpendicular to the longitudinal axis 8 of the catheter.

Due to the distal ends 5 of the optical fibres being closely packed, the light beam, which is emitted when a laser light source is connected, forms an essentially continuous bundle, for example a circular bundle, which is capable of ablating a continuous piece of tissue from a target vessel.

Referring to FIGS. 2a-2d, an ETS-anastomosis procedure with the above catheter will be described.

FIG. 2a shows a first step. The distal end 32 of the graft vessel 16 is attached to the side wall of the target vessel 21, leaving the part 42 (see FIG. 2b) of the wall tissue of the . target vessel 21 in front of the lumen of the graft vessel 16 intact so that the blood flow in the target vessel 21 can be left undisturbed. An advantage may be that attaching the graft to the target vessel does not require occlusion of the target vessel. The graft vessel 16 can be fixed to the target vessel 21 by any suitable connection technique, such as connection techniques known from the prior art which preferably do not require the part 42 of wall tissue to be removed before. FIG. 2a shows for example a suture 23 enclosing the ring member 15 as well as piercing through the graft vessel 16 and the target vessel 21. Instead of a suture 23 also a staple could be used. Further, as is shown in FIG. 2, the flanges 18 can be used for establishing a good connection to the target vessel 21. The flanges 18 can for example be glued to the target vessel 21.

After a firm and sufficiently leak tight connection between the graft vessel 16 and target vessel 21 has been established, the laser catheter 1 of FIG. 1 is inserted into the proximal end 31 of the graft vessel 16, see FIG. 2b. As can be seen in FIG. 2b, the bulge 22 on the outer circumference of the laser catheter 1 causes a similar bulge 24 in the wall of the graft vessel 16. This bulge 24 allows the surgeon to see how far the catheter is advanced in the graft vessel 16.

The laser catheter 1 is advanced distally (arrow D in FIG. 2b) until the light emitting surface 6 contacts the wall part 42 to be removed from the target vessel. In case not already done before, the channel 9 and optical fibres 4 are, subsequently, connected to a vacuum source 10 and laser light source , respectively. A vacuum is applied to the channel 9 and the laser procedure is started. Laser light is supplied to the optical fibres 4. Thus doing, the light emitting surface 6 gradually advances forward through the wall of the target vessel until said surface 6 faces or protrudes into the lumen 44 of the target vessel 21. The so called flap 14 is gripped by the suction mouth 11. At this moment, the laser procedure is finished and the laser light source can be switched off. Subsequently, the laser catheter is retracted in the direction opposite to arrow D, whilst the flap 14 is being removed by the suction gripper 11.

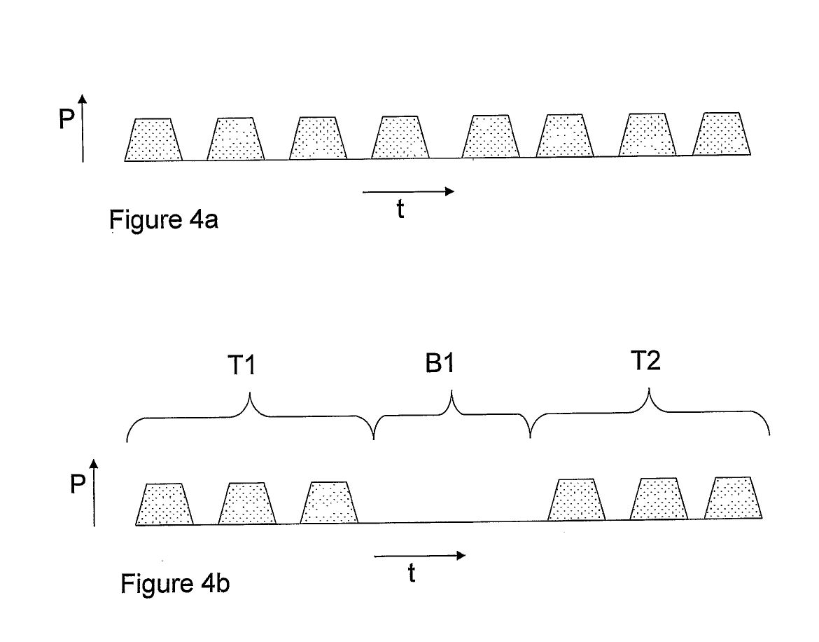

As soon as the laser catheter has been retracted over a sufficient distance, a clip 37 (FIG. 4d) or other closure is placed on the graft vessel 16 in order to close it off. Blood will be allowed to enter the graft vessel through the aperture 27, but will not be able to pass the clip 37. The proximal end 31 of the graft vessel can be connected by a ETE-anastomosis (ETE=End To End) to another vessel, such as another graft vessel, or it can be connected by an ETS-anastomosis to the same or another target vessel.

The laser catheter and its use as described up to here in the ‘detailed description’ corresponds essentially to the laser catheter disclosed in EP 750,476. Next, more specifically the present invention will be addressed.

Referring to FIGS. 1a and 1b, the optical fibres 4 have distal ends 5, which together define a light emitting surface 6. In FIG. 1b, the stop surface 7 of the bulge 22 can be seen around two rings of distal fibre ends 5 which together form the light emitting surface 6. According to the invention, the ablation power of the pulsating light beam emitted from the light emitting surface 6 is at least 40 mJ/mm² per pulse, or more preferably at least 45 mJ/mm² per pulse at the location of the light emitting surface. The power is measured at the location of the light emitting surface 6, where the light beam leaves the fibres. The area used for calculating the ablation power is the area of the light emitting surface, this being the sum of all the distal end areas of all optical fibres.

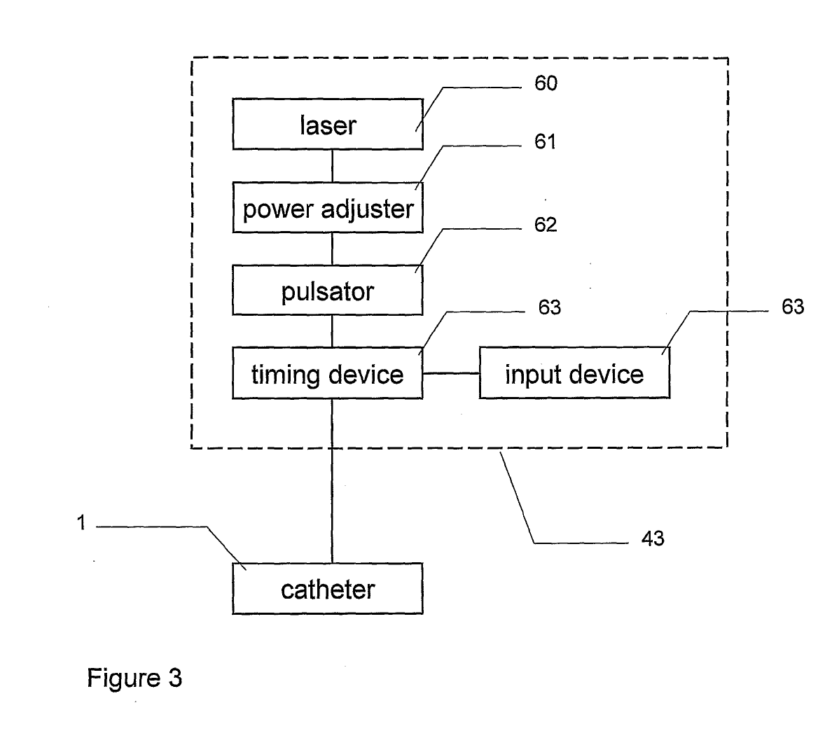

The power of the light beam can be adjusted in several ways. The laser apparatus itself can be provided with a power control for controlling the power of the lasers used. It is also possible that the transfer efficiency of the laser light through the fibres can be controlled, for example by changing the position or the angle between the laser emitting surface and the fibre receiving surface at the proximal side 41 of the fibres. In this way, the optical power of the laser is not completely transferred to the fibres. Yet another possibility would be to implement an optical dimmer in the optical fibres. In FIG. 3, a schematic view of the laser apparatus connected to the laser catheter 1 is presented, with a laser 60 and a power adjuster 61 depicted. The other functional parts of the assembly are discussed below.

In an embodiment, the catheter system comprises a laser catheter 1, a laser apparatus 60, an adjuster 61 for setting the ablation power of the pulsating light beam, a power sensor 65 for measuring the ablation power of the light beam and a controller 66 for controlling the adjuster 61 in response to the measurement of the power sensor 65.

When the catheter system is prepared for use, the laser apparatus 60 will first supply light with a laser power to the optical fibres. Because of the attenuation of the optical fibres and the adjuster 61, the ablation power at the location of the light emitting surface will be less than the laser power. To adjust the ablation power to its desired value, the catheter system is arranged for the following. First power sensor 65 measures the ablation power at the location of the light emitting surface. The controller 66 is arranged to compare this measured ablation power with a predefined power value requirement. A user of the catheter system, for example a surgeon, may act as the controller, when he or she adjusts the power of the laser apparatus manually after measuring the ablation power and reading the measured ablation power for example on a display. In case the measured ablation power is different from the predefined power value requirement, the controller, such as the user, operates the adjuster in such a manner that the attenuation of the adjuster is changed and that ablation power is increased when the measured ablation power is lower than the predefined power value requirement and that the ablation power is decreased when the measured ablation power is higher than the predefined power. Of course, the controller can also be an automatic device, for example implemented on a microprocessor.

In an embodiment according to the invention, the catheter system repeats the steps of measuring, comparing and adjusting until the measured ablation power meets the predefined power value requirement.

According to the invention, a high effectiveness of the surgery can be achieved when the predefined power value requirement is at least 40 mJ/mm² per pulse, preferably in the range of 40-100 mJ/mm² per pulse, or more preferably in the range of 40-60 mJ/mm² per pulse.

In FIG. 4a, a diagram shows, very schematically, the ablation power emitted at the location of the light emitting surface according to the invention, in the direction indicated by E as function of the time in the direction indicated by t. The power is emitted during very short pulsating intervals. In a preferred embodiment the pulsating frequency is at least 25 Hz, preferably in the range of 25-80 Hz.

The pulse width of the pulsating light beam is measured at full width at half maximum (F.W.H.M.) and is according to an embodiment in the range of 50-300 ns at F.W.H.M.

In another embodiment of the invention, the catheter system is provided with a timing device 61 and is arranged to emit the tubular bundle of light beam during at least two emitting intervals, separated with a break interval. The power of the light emitted according to this embodiment as function of the time is depicted very schematically in FIG. 4b. During the first interval T1 pulsating light is emitted. Only a few pulses are shown in FIG. 4b, in practice the number of pulses will be much larger in a interval. During the break interval B1, no light is emitted. And during the second interval T2, pulsating light is emitted again. Again, only a few pulses are shown in FIG. 4b, in practice the number of pulses will be much larger in a interval.

In a further embodiment the timing device 63 is further connected with an input device 64 for inputting time values for the emitting intervals and break intervals. This can be done for example by a numeric keyboard.

The invention is further described by the following clauses:
1] A catheter system comprising a laser catheter provided with a fibre bundle of optical fibres having distal ends defining a light emitting surface for emitting a light beam in the distal direction of the catheter; and a laser apparatus, comprising one or more lasers for supplying light to the optical fibres, wherein the system is preset or adjusted for emitting a pulsating light beam with an ablation power of at least 40 mJ/mm² per pulse at the location of the light emitting surface, or more preferably 45 mJ/mm² per pulse at the location of the light emitting surface,
2] A catheter system comprising a laser catheter provided with a fibre bundle of optical fibres having distal ends defining a light emitting surface for emitting a pulsating light beam in the distal direction of the catheter; a laser apparatus, comprising one or more lasers for supplying light to the optical fibres, and an adjuster for setting an ablation power of the pulsating light beam; a power sensor for measuring the ablation power and for generating a power signal representative for the measured ablation power; and, a controller for controlling the adjuster in response to the power signal to set said ablation power at least 40 mJ/mm² per pulse at the location of the light emitting surface, or more preferably 45 mJ/mm² per pulse at the location of the light emitting surface.
3] The catheter system according to one of the preceding clauses, wherein the ablation power is in the range of 40-100 mJ/mm² per pulse, preferably in the range of 40-60 mJ/mm² per pulse.
4] The catheter system according to one of the preceding clauses, wherein the optical fibres of the fibre bundle are arranged in a tubular configuration to define said light emitting surface as being ring shaped.
5] The catheter system according to one of the preceding clauses, further comprising a timing device for setting emitting intervals and break intervals at predefined time values, wherein the catheter system is arranged to emit the pulsating light beam during at least two emitting intervals, wherein each two consecutive intervals are separated by a break interval, during which emitting the pulsating light beam is interrupted.
6] The catheter system according to clause 5, wherein the timing device further comprises an input device for inputting predefined time values for the emitting intervals and break intervals.
7] The catheter system according to one of clauses 5-6, wherein the emitting intervals are each at least 2 s.
8] The catheter system according to one of clauses 5-7, wherein the emitting intervals are each in the range of 2-5 s.
9] The catheter system according to one of clauses 5-8, wherein the break interval is at least 1 s.
10] The catheter system according to one of clauses 5-9, wherein the break interval is in the range of 1-5 s.
11] The catheter system according to one of the preceding clauses, wherein the pulsating light beam has a pulsating frequency of at least 25 Hz, preferably in the range of 25-80 Hz, or more preferably around 40 Hz.
12] The catheter system according to one of the preceding clauses, wherein the pulsating light beam has a pulse width in the range of 50-300 ns at F.W.H.M.
13] The catheter system according to one of the preceding clauses, wherein the distal part of the catheter is provided with a gripper for gripping tissue.
14] The catheter system according to clause 13, wherein the gripper comprises a hollow channel extending within the fibre bundle of optical fibres and connectable to a vacuum source, the distal end of the channel defining a suction mouth.
15] The catheter system according to clause 14, wherein the suction mouth is arranged at a distance proximally from the light emitting surface and defines a suction surface parallel to the light emitting surface.
16] The catheter system according to one of the preceding clauses, further comprising a stop surface extending around the fibre bundle of optical fibres and facing in the distal direction, the stop surface being arranged at a distance proximally from the light emitting surface.
17] The catheter system according to clause 16, further comprising a ring member having dimensions adapted for, on the one hand, insertion of the distal end of the fibre bundle of optical fibres through said ring member and for, on the other hand, preventing passage of the stop surface through said ring member.
18] The catheter system according to clause 17, further comprising a graft vessel having diameter dimensions allowing, on the one hand, passage of the laser catheter and, on the other hand, insertion through said ring member.
19] The catheter system according to clause 18, wherein one end of the graft vessel is inserted through said ring member and folded back over said ring member and wherein the angle between the graft vessel and the part of the graft vessel folded back is in the range 90-180 degrees.
20] The catheter system according to one of the preceding clauses, wherein the light emitting surface is essentially perpendicular to the longitudinal direction of the catheter.
21] Method for preparing a catheter system according to one of the previous clauses for bypass surgery, comprising the step of:
a) measuring said ablation power at the location of the light emitting surface;
b) comparing the measured ablation power with a predefined power value requirement;
c) in case the measured ablation power is different from the predefined power value requirement, adjusting the ablation power of the pulsating light beam, emitted by the catheter system from the light emitting surface, to meet said predefined power value requirement.
22] Method according to clause 21, wherein the predefined power value requirement is at least 40 mJ/mm² per pulse, preferably in the range of 40-100 mJ/mm² per pulse, or more preferably in the range of 40-60 mJ/mm² per pulse.
23] Method according to clause 21 or 22, wherein steps a), b) and c) are repeated until the measured ablation power meets the predefined power value requirement.
24] Method according to one of clauses 21-23, further comprising the steps of:
d) setting at least two emitting intervals, during which a pulsating light beam is emitted, to at least 2 s, preferably in a range of 2-5 s, each two consecutive intervals being separated by a break interval, and,
e) setting the break intervals, during which emitting the pulsating light beam is interrupted, to at least 1 s, preferably in a range of 1-5 s.
25] Method according to one of clauses 21-24, further comprising the step of:
f) adjusting a pulsating frequency of the pulsating light beam, to at least 25 Hz, preferably in the range of 25-80 Hz, and more preferably around 40 Hz.
26] Method according to one of the clauses 21-25, further comprising the step of:
k) inserting a laser catheter into a graft vessel.
27] Method according to one of clauses 21-26, further comprising the step of:
l) inserting the graft vessel through a ring member.
28] Method according to one of clauses 21-27, further comprising the step of:
m) folding back one end of the graft vessel over the ring member wherein the angle between the graft vessel and the part of the graft vessel folded back is in the range 90-180 degrees.
29] Method according to one of clauses 27-28, further comprising the step of:
n) fasten the ring member onto the graft vessel.
30] Laser apparatus adjusted for supplying a pulsating light beam into optical fibres with an ablation power of at least 40 mJ/mm² per pulse, for ablating in an end-to-side anastomosis a passage in a side wall of a target vessel.
31] Method for preparing an catheter system according to one of the previous clauses for bypass surgery, comprising the steps of:
a) adjusting the ablation power of the pulsating light beam, emitted by the catheter system from the light emitting surface, to at least 40 mJ/mm² per pulse, preferably in the range of 40-100 mJ/mm² per pulse, or more preferably in the range of 40-60 mJ/mm² per pulse.
b) setting the emitting intervals to at least 2 s, preferably in a range of 2-5 s;
c) setting the break intervals to at least 1 s, preferably in a range of 1-5 s;
d) adjusting a pulsating frequency of the pulsating light beam to at least 25 Hz, preferably in the range of 25-80 Hz, or more preferably around 40 Hz.
32] Method for preparing a catheter system according to clause 31, further comprising the steps of :
g) inserting the laser catheter in a graft vessel;
h) inserting the graft vessel through the ring member;
i) fasten the ring member onto the graft vessel;
j) folding one end of the graft vessel over the ring member;
wherein the angle between the graft vessel and the part of the graft vessel folded back is in the range 90-180 degrees.
33] Method for preparing a catheter system according to one of clause 31-32, further comprising the step of:
aa) measuring the ablation power at the location of the light emitting surface.
34] Method for preparing a catheter system according to clause 33, further comprising the step:
ab) comparing a measured ablation power with a predefined ablation power.
35] Method for attaching a graft vessel end-to-side to a target vessel, using a catheter system according to one of the preceding clauses, comprising of:
a) inserting a graft vessel into a ring member;
b) folding back one end of the graft vessel over the ring member;
c) placing the fold of the graft vessel against the side wall of the target vessel and fixating the graft vessel to the target vessel;
d) inserting a laser catheter according to one of the previous clauses in a graft vessel;
e) positioning the laser catheter on the wall of the target vessel;
f) ablating a piece in the wall of the target vessel with the pulsating light beam and thereby cutting a piece of the wall part;
g) removing the piece of the wall part with a gripper;
wherein an ablation power of the pulsating light beam is at least 40 mJ/mm² per pulse at the location of the light emitting surface.
36] The catheter system according to one of clauses 1-20, wherein the laser apparatus is arranged to provide ultraviolet light, preferably having a wavelength in the range of 240-340, or about 308 nm.
37] Laser apparatus according to clause 30, wherein the laser apparatus is arranged to provide ultraviolet light, preferably having a wavelength in the range of 240-340, or about 308 nm.
38] Method according to one of clauses 21-29 or 31-35, wherein the pulsating light beam provides ultraviolet light, preferably having a wavelength in the range of 240-340, or about 308 nm.

Referring to the preceding explanation of the invention, it is noted that further modifications and embodiments are very well conceivable. Also further modifications and embodiments are within the scope of this invention.

1. A catheter system comprising:
a laser catheter provided with a fibre bundle of optical fibres having distal ends defining a light emitting surface for emitting a pulsating light beam in a distal direction of the catheter;
a laser apparatus, comprising one or more lasers for supplying light to the optical fibres, wherein the system is configured to emit the pulsating light beam with an ablation power of at least 40 mJ/mm² per pulse at the location of the light emitting surface; and
a stop surface extending around the fibre bundle of optical fibres and facing in the distal direction, the stop surface being arranged at a distance proximally from the light emitting surface.

2. The catheter system of claim 1, comprising:
an adjuster configured to set an ablation power of the pulsating light beam;
a power sensor configured to measure the ablation power and generate a power signal representative for the measured ablation power; and
a controller configured to control the adjuster in response to the power signal to set said ablation power.

3. The catheter system of claim 1, wherein the ablation power is in the range of 40-100 mJ/mm² per pulse.

4. The catheter system of claim 1, wherein the optical fibres of the fibre bundle are arranged in a tubular configuration to define said light emitting surface as being ring shaped.

5. The catheter system of claim 1, comprising a timing device configured to set emitting intervals and break intervals at predefined time values, wherein the catheter system is configured to emit the pulsating light beam during at least two emitting intervals, wherein each of two consecutive intervals are separated by a break interval, during which emitting the pulsating light beam is interrupted.

6. The catheter system of claim 5, wherein the timing device includes an input device configured to input predefined time values for the emitting intervals and break intervals.

7. The catheter system of claim 6, wherein the emitting intervals are each at least two seconds.

8. The catheter system of claim 6, wherein the emitting intervals are each in the range of two to five seconds.

9. The catheter system of claim 6, wherein the break interval is at least one second.

10. The catheter system of claim 6, wherein the break interval is in the range of one to five seconds.

11. The catheter system of claim 1, wherein the pulsating light beam includes a pulsating frequency of at least 25 Hz.

12. The catheter system of claim 1, wherein the pulsating light beam includes a pulse width in the range of 50-300 nanoseconds at full width at half maximum.

13. The catheter system of claim 1, wherein a distal part of the catheter with includes a gripper configured to grip tissue.

14. The catheter system of claim 13, wherein the gripper comprises a hollow channel extending within the fibre bundle of optical fibres, the hollow chamber configured to connect to a vacuum source, a distal end of the channel defining a suction mouth.

15. The catheter system of claim 14, wherein the suction mouth is arranged at a distance proximally from the light emitting surface and defines a suction surface parallel to the light emitting surface.

16. The catheter system of claim 1, wherein the stop surface is rigidly and integrally formed with a casing of the catheter.

17. The catheter system of claim 16, comprising a ring member including dimensions adapted for insertion of the distal end of the fibre bundle of optical fibres through said ring member and for inhibiting passage of the stop surface through said ring member.

18. The catheter system of claim 17, comprising a graft vessel including diameter dimensions allowing passage of the laser catheter and insertion through said ring member.

19. The catheter system of claim 18, wherein the graft vessel includes an artificial graft vessel.

20. The catheter system of claim 18, wherein one end of the graft vessel is configured to be inserted through said ring member and folded back over said ring member, and wherein the angle between the graft vessel and the part of the graft vessel folded back is in the range 90-180 degrees.

21. The catheter system of claim 1, wherein the light emitting surface is substantially perpendicular to a longitudinal direction of the catheter.

22-32. (canceled)
